(19) United States
(12) Patent Application Publication
(10) Pub. No.: US 2023/0035343 A1
(43) Pub. Date: Feb. 2, 2023

(54) METHOD FOR PREDICTING ONSET OF CHORIOAMNIONITIS

(71) Applicants: Shingo MIYAMOTO, Fukuoka (JP); Daichi URUSHIYAMA, Fukuoka (JP); Kenichiro HATA, Tokyo (JP)

(72) Inventors: Shingo MIYAMOTO, Kasuga-shi, Fukuoka (JP); Daichi URUSHIYAMA, Fukuoka-shi, Fukuoka (JP); Fusanori YOTSUMOTO, Fukuoka-shi, Fukuoka (JP); Kenichiro HATA, Chiyoda-ku, Tokyo (JP)

(73) Assignees: Shingo MIYAMOTO, Fukuoka (JP); Daichi URUSHIYAMA, Fukuoka (JP); Kenichiro HATA, Tokyo (JP)

(21) Appl. No.: 17/782,059

(22) PCT Filed: Dec. 4, 2020
(86) PCT No.: PCT/JP2020/045298

§ 371 Date: Jun. 2, 2022

(30) Foreign Application Priority Data
Dec. 6, 2019 (JP) 2019-221539

(51) Int. Cl. C12Q 1/689; C12Q 1/6869

(52) U.S. Cl. CPC C12Q 1/689; C12Q 1/6869

(57) ABSTRACT

Provided is a method for predicting the onset of chorioamnionitis, which may predict the onset of chorioamnionitis with high sensitivity. The method including obtaining a base sequence data group of a 16S ribosomal RNA gene in a sample from the vagina of a subject; detecting the presence of a bacterium selected from a bacterial group represented by G1 below based on the data group obtained; and associating the sample with the likelihood of onset of chorioamnionitis based on the number of bacterial species detected. The bacterial group G1: Finegoldia magna; Streptococcus anginosus; Aerococcus christensenii; Lactobacillus jensenii; Ureaplasma parvum; Prevotella disiens; Lactobacillus vaginalis; Prevotella buccalis; Dialister micraerophilus; Atopobium vaginae; Prevotella bivia; Prevotella amnii; Anaerococcus lactolytcus; Streptococcus agalactiae; Anaerococcus tetradius; Bacteroides fragilis; Gardnerella vaginalis; Mycoplasma hominis; and Sneathia sanguinegens.

### TECHNICAL FIELD

The present invention relates to a method for predicting the onset of chorioamnionitis.

### BACKGROUND ART

Chorioamnionitis is an inflammatory disease caused by infection of an fetal membrane, which is a fetal appendage, with bacteria. Chorioamnionitis is a condition that causes preterm birth and is closely related to the prognosis of a child after birth, such as a central nervous system disorder, or a chronic respiratory disorder. Diagnosis of chorioamnionitis is confirmed by placental pathology after delivery, and during pregnancy, diagnosis is made clinically and by using a marker for preterm delivery, such as granulocyte elastase. However, the sensitivity of these methods for detecting chorioamnionitis is low, which makes early detection difficult.

In relation to the above, a method for detecting chorioamnionitis by detecting a specific microorganism in an amniotic fluid has been proposed (see, for example, JP No. 2017-209063A).

### SUMMARY OF INVENTION

### Technical Problem

Since many cases of chorioamnionitis lead to preterm delivery in a short period of time after the onset of chorioamnionitis, there is a need for a method to predict the onset of choricamnionitis in a subject with high sensitivity. Therefore, an object of the present invention is to provide a method for predicting the onset of chorioamnionitis that may predict the onset of chorioamnionitis with high sensitivity.

### Solution to Problem

A first embodiment is a method for predicting the onset of chorioamnionitis, the method including: obtaining a base sequence data group of a 16S ribosomal RNA gene in a sample from the vagina of a subject; detecting the presence of a bacterium selected from a bacterial group represented by G1 below based on the data group obtained; and associating the sample with the likelihood of onset of chorioamnionitis based on the number of bacterial species detected.

Bacterial Group G1
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae;
Anaerococcus tetradius;
Bacteroides fragilis;
Gardnerella vaginalis;
Mycoplasma hominis; and
Sneathia sanguinegens.

A second embodiment is a method for detecting chorioamnionitis, the method including:—obtaining a base sequence data group of a 16S ribosomal RNA gene contained in a sample from the vagina of a subject; detecting the presence of a bacterium selected from the bacterial group represented by above-described G1 based on the data group obtained; and associating the sample with the presence of chorioamnionitis based on the number of bacterial species detected.

A third embodiment is a method for predicting or detecting the onset of chorioamnionitis, the method including: preparing a group of chorioamnionitis-associated bacteria by selecting choricamnionitis-associated bacteria in a target group; obtaining a base sequence data group of a 16S ribosomal RNA gene contained in a sample from the vagina of a subject; detecting the presence of a bacterium selected from the group of choricamnionitis-associated bacteria based on the data group obtained; and associating the sample with the likelihood of onset of chorioamnionitis or associating with the sample with the presence of chorioamnionitis based on the number of bacterial species detected.

### Advantageous Effects of Invention

According to the present invention, a method for predicting the onset of chorioamnionitis that may predict the onset of chorioamnionitis with high sensitivity may be provided.

### DESCRIPTION OF EMBODIMENTS

The term “step” as used herein encompasses not only an independent step, but also a step in which an anticipated effect of this step is achieved, even if the step cannot be clearly distinguished from another step. Hereinafter, embodiments of the present invention will be described in detail. However, the embodiments described below are illustrative of a method for predicting the onset of chorioamnionitis to embody the technical concept of the invention, and the present invention is not limited to the method for predicting the onset of chorioamnionitis described below.

### Method for Predicting Onset of Chorioamnionitis

A method for predicting the onset of chorioamnionitis includes: a first step of obtaining a base sequence data group of a 16S ribosomal RNA gene in a sample from the vagina of a subject; a second step of detecting the presence of a bacterium selected from a bacterial group represented by G1 below based on the data group obtained; and a third step of associating the sample with a likelihood of onset of chorioamnionitis based on the number of bacterial species detected.

Bacterial Group G1
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae;
Anaerococcus tetradius;
Bacteroides fragilis;
Gardnerella vaginalis;
Mycoplasma hominis; and
Sneathia sanguinegens.

In a method of using an amniotic fluid as a sample and analyzing a bacterial flora contained in the amniotic fluid, a bacterium cannot be detected unless the condition has progressed from chorioamnionitis to infection of the amniotic fluid. Therefore, although the presence of chorioamnionitis can be detected prenatally in a subject who has developed chorioamnionitis, it is difficult to predict the likelihood of the onset of chorioamnionitis in a subject who has not developed chorioamnionitis. Further, collecting an amniotic fluid is invasive and difficult to perform, and collecting an amniotic fluid a plurality of times is even riskier and more difficult to perform. On the other hand, in a method for predicting the onset of chorioamnionitis, by analyzing the bacterial flora contained in a sample from the vagina of a subject, the likelihood of the onset of chorioamnionitis can be predicted based on the presence or absence of a specific bacteria. Since a bacterial species to be detected is selected by associating a sample from a vagina with a confirmed diagnosis by placental pathology examination after delivery, as described below, the likelihood of the onset of chorioamnionitis may be predicted with high sensitivity.

### First Step

In a first step, a base sequence data group of a 16S ribosomal RNA gene contained in a sample from the vagina of a subject is obtained. The subject in the method for predicting the onset of chorioamnionitis may be any human pregnant woman, such as a pregnant woman in the perinatal period, or a pregnant woman in the perinatal period who is at risk of an imminent preterm delivery. A sample from a vagina may be, for example, a vaginal swab. A vaginal swab may be collected by employing a method commonly used with a commercially available kit, such as Opti-Swab Transport System (Puritan) or Catch-All Sample Collection Swab (Epicentre). The collected sample may be stored at −80° C., for example.

A base sequence data group of a 16S ribosomal RNA gene in a sample may be obtained, for example, by the following analytical method. A method including: extracting DNA from a sample; amplifying the extracted DNA by PCR using a universal primer set for a 16S ribosomal RNA gene to obtain an amplicon (PCR amplification product) of the 16S ribosomal RNA gene; and determining randomly the base sequence of the 16S ribosomal RNA gene contained in the amplicon may be used to obtain a data group of the sequence of the 16S ribosomal RNA gene.

DNA extraction from a sample may be carried out by a DNA extraction method commonly used in the art. For example, cells may be physically crushed and lysed by bead processing using glass beads, and DNA contained in a sample may be extracted using a commercially available DNA extraction kit.

The extracted DNA may be used as a template to amplify the 16S ribosomal RNA gene amplicon by PCR using a universal primer set for the 16S ribosomal RNA gene. The target region for amplification of the universal primer set for the 16S ribosomal RNA gene may be any of all variable regions from V1 to V9 of the 16S ribosomal RNA gene, and for example, the conserved region between the V1 region and the V2 region may be used as the target region. This makes it easy to obtain valid phylogenetic information from the 16S ribosomal RNA gene. Here, the universal primer may contain a so-called barcode sequence or the like, and the barcode sequence may be located at either the 5′ end or the 3′ end.

By randomly determining a base sequence of a 16S ribosomal RNA gene in the resulting amplicons, a base sequence data group is obtained. The base sequence data group is a combination of the base sequence of a 16S ribosomal RNA gene specific to a certain bacterial species and the amount (for example, number of reads) of an oligonucleotide having the base sequence. Here, “randomly determining” means determining any base sequence contained in a mixture of DNA or the like whose base sequence is to be determined as randomly as possible, without selecting only a certain sequence or eliminating a certain sequence from the mixture. When a base sequence is determined randomly in such a manner, DNA with a base sequence that has a high concentration in the mixture of extracted DNA or the like will be subjected to sequencing more frequently, while DNA with a base sequence that has a low concentration will be subjected to sequencing less frequently. On the other hand, a vaginal flora in a sample from a vagina contains a plurality of types of bacteria, and each type of bacteria has a unique base sequence on the 16S ribosomal RNA gene. Thus, in a base sequence data group obtained by randomly determining the base sequence of a mixture of DNA or the like prepared from a sample from a vagina, the number of reads that match the base sequence that may be arbitrarily selected (for example, one that matches a base sequence specific to a certain bacterial species) will reflect the abundance of bacteria constituting a vaginal microflora. In other words, the base sequence data group will reflect the structure of the vaginal flora.

The base sequence of a 16S ribosomal RNA gene in an amplicon is determined using a so-called next generation sequencer (NGS). Next generation sequencer is a term that is used in contrast to a capillary sequencer that uses the Sanger method. A next generation sequencer uses a sequencing principle such as a synthetic sequencing method, a pyrosequencing method, or a ligase reaction sequencing method. Specific examples of the next generation sequencer include a MiSeq (registered trademark) system (illmina), a HiSeq (registered trademark) system (illmina), and an IonPGM (registered trademark) system (Life Technology). Amplicon sequencing using a next generation sequencer can be performed according to the manufacturer's recommended protocol. In a sequencing analysis, low quality reads and other reads are removed by quality control, and randomly selected reads may be used as a base sequence data group.

In the first step, in place of a comprehensive analysis using a next generation sequencer, a quantitative PCR may be performed using a primer set specific for each of the bacterial species constituting the above-described bacterial group to obtain a base sequence data group of the 16S ribosomal RNA gene in a sample from a vagina. A primer set specific for each bacterial species can be created based on the standard sequence of the 16S ribosomal RNA gene for each bacterial species obtained from the RDP (Ribosomal Database Project, http://rdp.cme.msu.edu). Specifically, multiple alignments are performed using MEGA (Molecular Evolutionary Genetics Analysis, http://www.megasoftware.net) to identify a base sequence in the 16S ribosomal RNA gene that is common within a bacterial species and differ among species. Next, a primer set is designed for a base sequence to obtain a primer set specific to each bacterial species. The specificity of a designed primer set may be confirmed by in-silico PCR. Examples of the specific primer set include the following primer sets.

TABLE 1

| | Target bacterial species | | |
|---|---|---|---|
| | Finegoldia | Streptococcus | Aerococcus |
| | magna | anginosus | christensenii |
| Forward | CGGTCAAAG | GTATGTAAC | GAACAAACTT |
| primer | ATTTATCGG | ACATGTTAG | AGAAAGATGG |
| | TC | ATGC | SEQ ID |
| | SEQ ID | SEQ ID | NO: 5 |
| | NO: 1 | NO: 3 | |
| Reverse | GACAGAACT | GAACTTTCC | GAGCCGTT |
| primer | TTACGATAC | ATTCTCACA | ACCTCGCC |
| | G | CTCG | AACTAGT |
| | SEQ ID | SEQ ID | SEQ ID |
| | NO: 2 | NO: 4 | NO: 6 |
| Amplicon | 237 bp | 299 bp | 81 bp |
| size | | | |

Quantitative PCR can be performed by any method that can quantitatively obtain PCR amplification products, and a known method such as a real-time PCR or a digital PCR can be used. In this case, a base sequence data group is a combination of a base sequence of a region to be amplified in a specific primer set and the amount (for example, copy number) of an oligonucleotide having the base sequence, which is considered to be substantially the same as a base sequence data group obtained by a next generation sequencer.

### Second Step

In a second step, based on a base sequence data group to be obtained, the presence of a specific bacterium selected from the bacterial group is detected. By this step, the number of bacterial species of a specific bacterium in a sample is identified. The presence of a specific bacterium in a sample may be determined based on the amount of oligonucleotides having the base sequence of a 16S ribosomal RNA gene specific to the bacterial species. In cases in which a base sequence data group is obtained from a next generation sequencer, when the number of reads of oligonucleotides with a specific base sequence or a corrected number of reads of oligonucleotides with a specific base sequence corrected by the number of reads of all bacterial species is equal to or greater than a predetermined cutoff value, it may be determined that a bacterial species corresponding to a specific base sequence is present. In cases in which a base sequence data group is obtained by quantitative PCR, when a corrected value, which is the copy number of oligonucleotides with a specific base sequence corrected by the copy number of all bacterial species, is equal to or greater than a predetermined cutoff value, it may be determined that a bacterial species corresponding to a specific base sequence is present.

The above-described bacterial group may, for example, be composed of a bacterial species selected by the selection method for chorioamnionitis-associated bacteria described below. Detection of a specific bacterium selected from the above-described bacterial group can predict the onset of chorioamnionitis with high sensitivity. A bacterial group that specific bacteria constitute may be one of G2, G3, or G4 below, or another bacterial group selected by the selection method for chorioamnionitis-associated bacteria described below. Higher YoudenIndex and odds ratios may be achieved by using the bacterial group represented by G2. Higher specificity and odds ratio may be achieved by using the bacterial group represented by G3. Furthermore, the bacterial group represented by G4 may be used in combination with the bacterial group represented by G1 or G2. By employing a different bacterial group selected by the selection method for chorioamnionitis-associated bacteria, which is described below, a more appropriate approach may be taken to target groups with different races, lifestyles, diseases, complications, or the like.

Bacterial Group G2
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae; and
Anaerococcus tetradius.

Bacterial Group G3
Finegoldia magna;
Streptococcus anginosus;
Aerococcus christensenii;
Lactobacillus jensenii;
Ureaplasma parvum;
Prevotella disiens;
Lactobacillus vaginalis; and
Prevotella buccalis.

Bacterial Group G4
Finegoldia magna;
Streptococcus anginosus;
Aerococcus christensenii;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Anaerococcus tetradius.

### Third Step

In a third step, based on the number of specific bacterial species detected in a sample, the sample is associated with the likelihood of onset of chorioamnionitis in a subject from whom the sample is obtained. The number of bacterial species used for association is, for example, two or more, and may be two or three. For example, when two or more types of specific bacteria are detected in a sample, a high risk of onset of chorioamnionitis is predicted in a subject from whom the sample is taken.

A method for predicting the onset of chorioamnionitis may be adjusted for the sensitivity, the accuracy, or the like, depending on a combination of a bacterial group from which a specific bacterium is selected and the number of species of specific bacteria detected in a sample. For example, the onset of chorioamnionitis may be predicted with high sensitivity by associating detection of two or more specific bacteria selected from the bacterial group represented by G1 above with the likelihood of onset of the disease. A bacterial group other than that represented by G1 may be used to predict the onset of chorioamnionitis as follows. For example, the onset of chorioamnionitis may be predicted with a high YoudenIndex and an excellent odds ratio by associating detection of three or more specific bacteria selected from the bacterial group represented in G2 above with the likelihood of onset of chorioamnionitis. Detection of two or more specific bacteria selected from the bacterial group represented by G3 above may be associated with the likelihood of onset of chorioamnionitis. Furthermore, a case in which two or more specific bacteria selected from the bacterial group represented by G4 above are detected may be associated with the likelihood of onset of chorioamnionitis. A case in which at least one of specific bacteria selected from the bacterial group represented by G4 above is detected and at least one of specific bacteria selected from the bacterial group represented by G1 or G2 above is detected may be associated with the likelihood of onset of chorioamnionitis.

For a subject who is predicted to be at high risk of onset of chorioamnionitis, an additional test such as an amniotic fluid test may be carried out, and if necessary, a treatment such as follow-up observation or therapeutic intervention may be carried out.

### Method for Detecting Choricamnionitis

A method for detecting chorioamnionitis includes: a first step of obtaining a base sequence data group of a 16S ribosomal RNA gene in a sample from the vagina of a subject; a second step of detecting the presence of a bacterium selected from a bacterial group represented by the above-described G1 based on the data group obtained; and a fourth step of associating the sample with the presence of chorioamnionitis based on the number of bacterial species detected.

Based on the number of specific bacterial species in a sample from a vagina, the presence of chorioamnionitis in a subject may be detected. This allows detection of the presence of chorioamnionitis in a subject without waiting for a definitive diagnosis by placental pathology after delivery, and allows therapeutic intervention or the like based on a diagnosis of the onset of chorioamnionitis. In other words, the method for detecting chorioamnionitis may be a method for diagnosing chorioamnionitis in a subject.

The first step and the second step in the method for detecting chorioamnionitis are the same as those in the method for predicting the onset of chorioamnionitis. In the fourth step, based on the number of bacterial species detected in a sample, the sample is associated with the presence of chorioamnionitis in a subject from which the sample is obtained. The number of bacterial species used for association is, for example, two or more, and may be two or three. For example, when two or more specific bacteria are detected in a sample, the presence of chorioamnionitis in a subject from which the sample was taken may be detected with high sensitivity.

A method for detecting the presence of chorioamnionitis may be adjusted for the sensitivity, the accuracy, or the like, depending on a combination of a bacterial group from which a specific bacterium is selected and the number of species of specific bacteria detected in a sample. For example, the presence of choricamnionitis may be detected with high sensitivity by associating detection of two or more specific bacteria selected from the bacterial group represented by G1 above with the presence of chorioamnionitis. A bacterial group other than that represented by G1 may be used to detect the presence of chorioamnionitis in a subject as follows. For example, the presence of chorioamnionitis may be detected with a high YoudenIndex and an excellent odds ratio by associating detection of three or more specific bacteria selected from the bacterial group represented in G2 above with the presence of chorioamnionitis. For example, detection of two or more specific bacteria selected from the bacterial group represented by G3 above may be associated with the presence of chorioamnionitis. For example, a case in which two or more specific bacteria selected from the bacterial group represented by G4 above are detected may be associated with the presence of chorioamnionitis. For example, a case in which at least one of specific bacteria selected from the bacterial group represented by G4 above is detected and at least one of specific bacteria selected from the bacterial group represented by G1 or G2 above is detected may be associated with the presence of chorioamnionitis.

### Method for Treating Chorioamnionitis

A method for treating chorioamnionitis may include: a first step of obtaining a base sequence data group of a 16S ribosomal RNA gene in a sample from the vagina of a subject; a second step of detecting the presence of a bacterium selected from a bacterial group represented by G1 below based on the data group obtained; a third step of associating the sample with the likelihood of onset of chorioamnionitis based on the number of bacterial species detected; and a fifth step of treating a subject at high risk of onset of chorioamnionitis.

By providing treatment as needed to a subject at high risk of onset of chorioamnionitis, an effect such as reduction of preterm birth, prolongation of gestation period, control of maternal infection, control of neonatal infection, control of a neonatal disease such as fetal inflammatory response syndrome, neonatal meningitis, neonatal chronic lung disease, periventricular leukomalacia, cerebral palsy, or developmental delay, control of incidence of disabilities, control of medical and social security costs, or promotion of social advancement of child-rearing generation can be obtained. Here, a treatment to a subject may be any treatment for chorioamnionitis, and examples thereof include a cure, an improvement, a control of progression (prevention of worsening), or a prevention of chorioamnionitis and an alleviation of a symptom caused by chorioamnionitis. Specific examples thereof include administration of an antibacterial agent, administration of a drug to inhibit an uterine contraction, administration of a steroid, administration of a labor-inducing drug, induction of labor, cesarean section, hysterotomy, removal of uterine contents, and hysterectomy.

### Method for Selecting Chorioamnionitis-Associated Bacteria

A method for selecting chorioamnionitis-associated bacteria includes: a sixth step of obtaining a base sequence data group of a 16S ribosomal RNA gene in a sample from the vagina of a subject included in a target group composed of a plurality of subjects; a seventh step of determining the Blanc classification of chorioamnionitis by placental pathology of the subject after delivery; an eighth step of machine learning an association between a base sequence data group of the subject and the Blanc classification to rank a bacterial species associated with chorioamnionitis; and a ninth step of selecting chorioamnionitis-associated bacteria based on the ranking.

By machine learning the relationship between the bacterial flora structure in a sample from a vagina of a target group and the diagnosis of chorioamnionitis by placental pathology, chorioamnionitis-associated bacteria that are associated with chorioamnionitis in the target group and are present in the vagina may be selected. By detecting selected chorioamnionitis-associated bacteria in a sample from the vagina of any given subject, the risk of onset of chorioamnionitis in the subject may be assessed. This allows reduction of preterm birth, prolongation of gestation period, control of neonatal infection associated with chorioamnionitis.

A target in the selection method is the same as a target in the above-described prediction method. A target group composed of a plurality of subjects may be a group of randomly selected subjects, or a group of subjects that exhibit a tendency to have similar vaginal flora. A group composed of subjects who show a tendency to have similar vaginal microflora may be a target group with a common race, a target group with a similar lifestyle, a target group with a similar disease, complication, or the like. Furthermore, a method for obtaining a base sequence data group of a 16S ribosomal RNA gene contained in a sample is the same as the first step described above.

In the seventh step, a pathological examination is conducted on the placenta obtained after delivery for each subject to make a definitive diagnosis of chorioamnionitis, and the Blanc classification is determined. Here, the Blanc classification is an index of the degree of pathologic inflammation of chorioamnionitis. In the eighth step, an association between a target base sequence data group and the Blanc classification is machine learned to identify bacterial species associated with chorioamnionitis and rank the association with the Blanc classification. For machine learning, a method appropriately selected from known methods, such as a random forest, can be applied. In the ninth step, chorioamnionitis-associated bacteria are selected based on the ranking. The bacterial species may be selected in the order of their association with chorioamnionitis. The number of bacterial species to be selected may be, for example, 30, 20, 15 or 3.

### EXAMPLES

Hereinafter, the present invention will be described more specifically by way of Examples, but the present invention is not limited to these Examples.

### Example 1

Sixty-six pregnant women with unruptured water who were admitted to Fukuoka University Hospital with a diagnosis of imminent preterm delivery over a two-year period beginning in May 2014 and for whom a sufficient volume of specimen could be collected for analysis were included as subjects. Written informed consent was obtained from the subjects. Placental pathology was conducted after delivery, and the subjects were divided into two groups: 40 in a chorioamnionitis group (Blanc classification, stage II or higher) and 26 in a non-chorioamnionitis group (Blanc classification, stage I or lower). Vaginal swabs collected from the subjects at the time of admission were stored at −80° C. as specimens.

Bacteria in each specimen were lysed with glass beads using Pathogen Lysis Tube L (QIAGEN), and DNA in each specimen was extracted using QIAamp UCP Pathogen Mini Kit (QIAGEN). Using the extracted DNA as a template, PCR amplification products were obtained by PCR reactions under the following conditions using the universal primer sets for the V1 and V2 regions of the 16S ribosomal RNA gene described below. The obtained PCR amplification products were stored at 4° C.

TABLE 2
Reaction mixture Composition

| | | |
|---|---|---|
| Template | 1 | μL |
| Forward primer (5 μM) | 2 | μL |
| Reverse primer (5 μM) | 2 | μL |
| 10 X Loading Buffer (Takara) | 2 | μL |
| dNTP (Takara) | 2 | μL |
| Taq polymerase | 0.2 | μL |
| Distilled water | 11.8 | μL |
| Total | 21.0 | μL |

TABLE 3

| | | |
|---|---|---|
| Forward | TCGTCGGCAGCGT | SEQ ID NO: 7 |
| primer | CAGATGTGTATAA | |
| | GAGACAGAGRGTT | |
| | TGATYMTGGCTCA | |
| | G | |
| Reverse | GTCTCGTGGGCTC | SEQ ID NO: 8 |
| primer | GGAGATGTGTATA | |
| | AGAGACAGTGCTG | |
| | CCTCCCGTAGGAG | |
| | T | |

TABLE 4
Reaction condition

| | |
|---|---|
| HOLD | 95° C., 3 min. |
| CYCLES | 95° C., 15 sec.; 55° C., 15 sec.; 72° C., 15 sec.; 35 Cycles |
| HOLD | 72° C., 3min. |

The PCR amplification products (amplicons) of 66 specimens obtained above were simultaneously analyzed for 16S amplicons using a next generation sequencer (NGS: MiSeq (registered trademark) sequencer) according to the protocol recommended by the manufacturer. Sequence data of PCR amplification products were obtained as Fastq file format. From the obtained reads, low quality reads were excluded by quality control, adapter sequences were deleted, and approximately 1,300 reads were randomly selected to obtain a base sequence data group. The obtained data group was divided into a chorioamnionitis group and a non-chorioamnionitis group, and subjected to machine learning (random forest) to identify the top 20 bacterial species most likely to be associated with chorioamnionitis. The identified bacterial species are listed below.

TABLE 5

| Bacterial species | Importance |
|---|---|
| Finegoldia magna | 6.91 |
| Streptococcus anginosus | 5.58 |
| Aerococcus christensenii | 4.85 |
| Pseudomonas sp. Ag1 | 4.12 |
| Peptoniphilus sp. BV3BML2 | 3.52 |
| Lactobacillus jensenii | 3.13 |
| Ureaplasma parvum | 2.74 |
| Prevotella disiens | 2.55 |
| Lactobacillus vaginalis | 2.50 |
| Prevotella buccalis | 2.31 |
| Lactobacillus gasseri | 2.21 |
| Dialister micraerophilus | 2.12 |
| Atopobium vaginae | 2.01 |
| Prevotella bivia | 1.82 |
| Peptoniphilus sp. 2002.2300004 | 1.59 |
| Prevotella amnii | 1.58 |
| Enterococcus avium | 1.42 |
| Anaerococcus lactolyticus | 1.41 |
| Streptococcus agalactiae | 1.27 |
| Anaerococcus tetradius | 1.03 |

Among these bacteria, 18 species were detected in the chorioamnionitis group except Lactobacillus gasseri and Enterococcus avium, and the following 15 species of bacteria were identified to bacterial species name. Hereafter, these are also referred to as a bacterial group G2.

TABLE 6
Bacterial group G2

Finegoldia magna
Streptococcus anginosus
Aerococcus christensenii
Lactobacillus jensenii
Ureaplasma parvum
Prevotella disiens
Lactobacillus vaginalis
Prevotella buccalis
Dialister micraerophilus
Atopobium vaginae
Prevotella bivia
Prevotella amnii
Anaerococcus lactolyticus
Streptococcus agalactiae
Anaerococcus tetradius

### Assessment of Risk of Onset of Chorioamnionitis

The relationship between the number of bacterial species detected in the specimens and the risk of onset of chorioamnionitis among the bacteria selected from the above-described bacterial group G2 was evaluated. When detection of two or more bacterial species belonging to the bacterial group G2 was determined to be positive (high risk of onset of chorioamnionitis), the sensitivity, the specificity, and the YoudenIndex were 68%, 69%, and 37%, respectively. When detection of three or more bacteria belonging to the bacterial group G2 was determined to be positive, the sensitivity, the specificity, and the YoudenIndex were 60%, 88%, and 48%, respectively. The results are summarized in the table below.

### Comparative Example 1

The risk of onset of chorioamnionitis was evaluated in the same manner, except that the following bacterial group G0, which was detected in the vagina among the bacteria detected in the amniotic fluid test described in Japanese Patent Application No. 2017-209063, was used instead of the above-described bacterial group G2. When detection of two or more bacterial species belonging to the bacterial group G0 was determined to be positive, the sensitivity, the specificity, and the YoudenIndex were 53%, 77%, and 29%, respectively. When detection of three or more bacteria belonging to the bacterial group G0 was determined to be positive, the sensitivity, the specificity, and the YoudenIndex were 18%, 100%, and 18%, respectively. The results are summarized in the table below.

TABLE 7
Bacterial group G0

Bacteroides fragilis
Gardnerella vaginalis
Mycoplasma hominis
Sneathia sanguinegens
Streptococcus agalactiae
Lactobacillus jensenii
Ureaplasma parvum

### Example 2

The union of the bacterial group G0 and the bacterial group G2 was designated as a bacterial group G1. The risk of onset of chorioamnionitis was evaluated in the same manner, except that the bacterial group G1 was used instead of the above-described bacterial group G2. When detection of two or more bacterial species belonging to the bacterial group G1 was determined to be positive, the sensitivity, the specificity, and the YoudenIndex were 78%, 58%, and 35%, respectively. When detection of three or more bacteria belonging to the bacterial group G1 was determined to be positive, the sensitivity, the specificity, and the YoudenIndex were 63%, 77%, and 39%, respectively. The results are summarized in the table below.

TABLE 8
Bacterial group G1

Finegoldia magna
Streptococcus anginosus
Aerococcus christensenii
Lactobacillus jensenii
Ureaplasma parvum
Prevotella disiens
Lactobacillus vaginalis
Prevotella buccalis
Dialister micraerophilus
Atopobium vaginae
Prevotella bivia
Prevotella amnii
Anaerococcus lactolyticus
Streptococcus agalactiae
Anaerococcus tetradius
Bacteroides fragilis
Gardnerella vaginalis
Mycoplasma hominis
Sneathia sanguinegens

### Example 3

The risk of onset of chorioamnionitis was evaluated in the same manner, except that the bacterial group G3 below was used instead of the above-described bacterial group G2. When detection of two or more bacterial species belonging to the bacterial group G3 was determined to be positive, the sensitivity, the specificity, and the YoudenIndex were 53%, 85%, and 37%, respectively. When detection of three or more bacteria belonging to the bacterial group G3 was determined to be positive, the sensitivity, the specificity, and the YoudenIndex were 35%, 100%, and 35%, respectively. The results are summarized in the table below.

TABLE 9
Bacterial group G3

Finegoldia magna
Streptococcus anginosus
Aerococcus christensenii
Lactobacillus jensenii
Ureaplasma parvum
Prevotella disiens
Lactobacillus vaginalis
Prevotella buccalis
Streptococcus agalactiae

### Example 4

The risk of onset of chorioamnionitis was evaluated in the same manner, except that the bacterial group G4 below was used instead of the above-described bacterial group G2. When detection of two or more bacterial species belonging to the bacterial group G4 was determined to be positive, the sensitivity, the specificity, and the YoudenIndex were 53%, 77%, and 29%, respectively. When detection of three or more bacteria belonging to the bacterial group G4 was determined to be positive, the sensitivity, the specificity, and the YoudenIndex were 35%, 96%, and 31%, respectively. The results are summarized in the table below.

TABLE 10
Bacterilal group G4

Finegoldia magna
Streptococcus anginosus
Aerococcus christensenii
Prevotella disiens
Lactobacillus vaginalis
Prevotella buccalis
Dialister micraerophilus
Atopobium vaginae
Prevotella bivia
Prevotella amnii
Anaerococcus lactolyticus
Anaerococcus tetradius

### Example 5

The risk of onset of chorioamnionitis was evaluated in the same manner, except that at least one species selected from the above-described bacterial group G4 and at least one species selected from the above-described bacterial group G2 were detected instead of the above-described bacterial group G2. When detection of two or more bacterial species belonging to the bacterial group G4 or the bacterial group G2 was determined to be positive, the sensitivity, the specificity, and the YoudenIndex were 65%, 73%, and 38%, respectively. When detection of three or more bacteria belonging to the bacterial group G4 or the bacterial group G2 was determined to be positive, the sensitivity, the specificity, and the YoudenIndex were 60%, 880%, and 37%, respectively. The results are summarized in the table below.

### Example 6

The risk of onset of chorioamnionitis was evaluated in the same manner, except that at least one species selected from the above-described bacterial group G4 and at least one species selected from the above-described bacterial group G1 were detected instead of the above-described bacterial group G2. When detection of two or more bacterial species belonging to the bacterial group G4 or the bacterial group G1 was determined to be positive, the sensitivity, the specificity, and the YoudenIndex were 68%, 73%, and 41%, respectively. When detection of three or more bacteria belonging to the bacterial group G4 or the bacterial group G2 was determined to be positive, the sensitivity, the specificity, and the YoudenIndex were 60%, 77%, and 37%, respectively. The results are summarized in the table below.

TABLE 11
Determining that 2 or more detected bacteria are positive

| | | | | Positive | Negative | |
|---|---|---|---|---|---|---|
| Bacterial | Sens- | Spec- | Youden | predictive | predictive | Odds |
| group | itivity | itivity | Index | value | value | ratio |
| G0 | 53% | 77% | 29% | 78% | 51% | 3.68 |
| G1 | 78% | 58% | 35% | 74% | 63% | 4.70 |
| G2 | 68% | 69% | 37% | 77% | 58% | 4.67 |
| G3 | 53% | 85% | 37% | 84% | 54% | 6.08 |
| G4 | 53% | 77% | 29% | 78% | 51% | 3.68 |
| G4 + G2 | 65% | 73% | 38% | 79% | 58% | 5.04 |
| G4 + G1 | 68% | 73% | 41% | 79% | 59% | 5.64 |

TABLE 12
Determining that 3 or more detected bacteria are positive

| | | | | Positive | Negative | |
|---|---|---|---|---|---|---|
| Bacterial | Sens- | Spec- | Youden | predictive | predictive | Odds |
| group | itivity | itivity | Index | value | value | ratio |
| G0 | 18% | 100% | 18% | 100% | 44% | ND |
| G1 | 63% | 77% | 39% | 81% | 57% | 5.56 |
| G2 | 60% | 88% | 48% | 89% | 59% | 11.50 |
| G3 | 35% | 100% | 35% | 100% | 50% | ND |
| G4 | 35% | 96% | 31% | 93% | 49% | 13.46 |
| G4 + G2 | 60% | 88% | 48% | 89% | 59% | 11.50 |
| G4 + G1 | 60% | 77% | 37% | 80% | 56% | 5.00 |

The use of bacterial groups G1 to G4 detected in a sample from a vagina was more sensitive in predicting the onset of chorioamnionitis than the use of bacterial group G0 detected in an amniotic fluid test. For example, detection of two or more bacteria selected from bacterial group G1 could predict the onset of chorioamnionitis with a sensitivity as high as 78%. By detecting three or more bacteria selected from bacterial group G2, a YoudenIndex of 48% could be achieved.

### Comparison of Positive Group and Negative Group for Prediction of Onset of Chorioamnionitis

The characteristics of the positive group and the negative group when using bacterial group G1 to predict the onset of chorioamnionitis are described below. Values are medians, and Mann-Witney U test and Fisher's exact test were used for significance tests. The number of onset of developmental disorders in 3-year-olds excludes cases with abnormalities including chromosomes.

TABLE 13

| | Positive | Negative | |
|---|---|---|---|
| | group | group | |
| | (N = 16) | (N = 27) | P value |
| Weeks of pregnancy | 30.4 | 29.9 | 0.257 |
| at the time of | | | |
| sampling (weeks) | | | |
| Cervical length at | 15.5 | 20.0 | 0.307 |
| the time of | | | |
| sampling (mm) | | | |
| Serum CRP level | 0.35 | 0.30 | 0.828 |
| (mg/dL) | | | |
| Histological | 14/16 (80%) | 11/27 (41%) | 0.004 |
| chorioamnionitis | | | |
| (Blanc classification | | | |
| stageII-III) | | | |
| Weeks of delivery | 31.6 | 33.9 | 0.010 |
| (weeks) | | | |
| Pregnancy extension | 10.6 | 26.1 | 0.002 |
| period (days) | | | |
| Birth weight (g) | 1591 | 1945 | 0.014 |
| Developmentdisorder | 5/14 (36%) | 0/25 (0%) | 0.003 |
| in 3-year-olds | | | |

The disclosure of Japanese Patent Application No. 2019-221539 (filing date: Dec. 6, 2019) are hereby incorporated by reference in its entirety. All Documents, patent applications, and technical standards described herein are incorporated by reference herein to the same extent as if each of the Documents, patent applications, and technical standards had been specifically and individually indicated to be incorporated by reference.

1. A method for predicting the onset of chorioamnionitis, the method comprising:
obtaining a base sequence data group of a 16S ribosomal RNA gene in a sample from the vagina of a subject;
detecting a presence of a bacterium selected from a bacterial group represented by G1 below based on the data group obtained; and
associating the sample with the likelihood of onset of chorioamnionitis based on a number of bacterial species detected, where
Bacterial group G1 is:
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae;
Anaerococcus tetradius;
Bacteroides fragilis;
Gardnerella vaginalis;
Mycoplasma hominis; and
Sneathia sanguinegens.

2. The method according to claim 1, comprising associating the sample with the likelihood of onset of chorioamnionitis when the number of bacterial species detected is two or more.

3. A method for detecting chorioamnionitis, the method comprising:
obtaining a base sequence data group of a 16S ribosomal RNA gene contained in a sample from the vagina of a subject;
detecting a presence of a bacterium selected from a bacterial group represented by G1 below based on the data group obtained; and
associating the sample with the presence of chorioamnionitis based on a number of bacterial species detected, where
Bacterial group G1 is:
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae;
Anaerococcus tetradius;
Bacteroides fragilis;
Gardnerella vaginalis;
Mycoplasma hominis; and
Sneathia sanguinegens.

4. The method according to claim 3, comprising associating the sample with the presence of chorioamnionitis when the number of bacterial species detected is two or more.

5. The method according to claim 1, comprising:
obtaining an amplicon of a 16S ribosomal RNA gene in the sample; and
randomly determining the base sequence of the 16S ribosomal RNA gene contained in the amplicon to obtain the data group.

6. The method according to claim 1, wherein the number of bacterial species detected is two or three.

7. The method according to claim 1, wherein the bacterial group is the bacterial group represented by G2, where
Bacterial group G2 is:
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae; and
Anaerococcus tetradius.

8. The method according to claim 3, comprising:
obtaining an amplicon of a 16S ribosomal RNA gene in the sample; and
randomly determining the base sequence of the 16S ribosomal RNA gene contained in the amplicon to obtain the data group.

9. The method according to claim 3, wherein the number of bacterial species detected is two or three.

10. The method according to claim 3, wherein the bacterial group is the bacterial group represented by G2, where
Bacterial group G2 is:
Finegoldia magna;
Streptococcus anginosus;
Aerococcus christensenii;
Lactobacillus jensenii;
Ureaplasma parvum;
Prevotella disiens;
Lactobacillus vaginalis;
Prevotella buccalis;
Dialister micraerophilus;
Atopobium vaginae;
Prevotella bivia;
Prevotella amnii;
Anaerococcus lactolytcus;
Streptococcus agalactiae; and
Anaerococcus tetradius.


SEQUENCE LISTING (8 sequences)

SEQ ID NO: 1 - DNA, 20 bp, Artificial Sequence
  F-primer
cggtcaaagatttatcggtc

SEQ ID NO: 2 - DNA, 19 bp, Artificial Sequence
  R-primer
gacagaactttacgatacg

SEQ ID NO: 3 - DNA, 22 bp, Artificial Sequence
  F-primer
gtatgtaacacatgttagatgc

SEQ ID NO: 4 - DNA, 22 bp, Artificial Sequence
  R-primer
gaactttccattctcacactcg

SEQ ID NO: 5 - DNA, 20 bp, Artificial Sequence
  F-primer
gaacaaacttagaaagatgg

SEQ ID NO: 6 - DNA, 23 bp, Artificial Sequence
  R-primer
gagccgttacctcgccaactagt

SEQ ID NO: 7 - DNA, 53 bp, Artificial Sequence
  F-primer
tcgtcggcagcgtcagatgtgtataagagacagagrgtttgatymtggctcag

SEQ ID NO: 8 - DNA, 53 bp, Artificial Sequence
  R-primer
gtctcgtgggctcggagatgtgtataagagacagtgctgcctcccgtaggagt